(19) United States
(12) Patent Application Publication
(10) Pub. No.: US 2004/0181080 A1
(43) Pub. Date: Sep. 16, 2004

(54) Epoxidation process using amine catalysts

(75) Inventors: Mauro Francesco Alessio Adamo, Sheffield (GB); Varinder Kumar Aggarwal, Sheffield (GB)

Correspondence Address: GLAXOSMITHKLINE, Corporate Intellectual Property - UW2220, P.O. Box 1539, King of Prussia, PA, 19406-0939, US

(73) Assignee: The University of Sheffield

(21) Appl. No.: 10/808,622

(22) Filed: Mar. 24, 2004

Related U.S. Application Data
(63) Continuation of application No. 10/395,409, filed on Mar. 24, 2003, now abandoned.
Application No. 10/395,409 is a continuation of application No. 09/890,298, filed on Sep. 27, 2001, now abandoned.
Application No. 09/890,298 is a 371 of international application No. PCT/GB00/00258, filed on Jan. 28, 2000.

(30) Foreign Application Priority Data
Jan. 29, 1999 (GB) 9902055.4

(51) Int. Cl. C07D 301/03

(52) U.S. Cl. 549/523

(57) ABSTRACT

A process for the epoxidation of an alkene, which process comprises reaction of an alkene with an oxidising agent in the presence of a catalyst, characterised in that the catalyst is an amine of formula (I), wherein T represents hydrogen or a moiety of formula (a); R¹, R², R³, R⁴, R⁵ and R⁶ each independently represents hydrogen, optionally substituted alkyl, an optionally substituted aryl group, heterocyclyl or an optionally substituted aralkyl group wherein substituents for the above mentioned groups are selected from up to three of alkyl, aryl, heterocycyl, hydroxy, alkoxy or a group NR^{s}R^{t} wherein R^{s} and R^{t} each independently represents hydrogen, alkyl or alkylcarbonyl and R⁷ represents hydrogen, alkyl, aryl or aralkyl; or T represents a moiety (a) wherein R¹ together with R² represents an optionally substituted alkylene chain comprising 2 to 6 carbon atoms the alkylene chain being optionally interrupted with an oxygen atom or a group NR^{p} wherein R^{p} is hydrogen or alkyl, and wherein optional substituents for any carbon atom of the alkylene chain are selected from hydroxy, alkoxy, oxo or a group NR^{s}R^{t} wherein R^{s} and R^{t} each independently represents hydrogen, alkyl or alkylcarbonyl or substituents on any two adjacent carbon atoms of the chain together with the carbon atoms to which they are attached form an alicyclic, aryl or heterocyclic ring; and R³, R⁴, R⁵, R⁶ and R⁷ are as defined above.

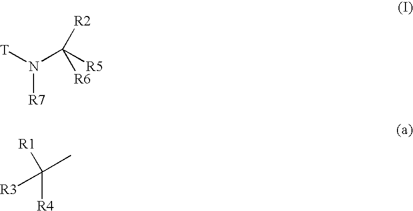

This invention relates to a novel process and in particular a process for the epoxidation of unfunctionalised alkenes.

The catalytic asymmetric epoxidation of unfunctionalised alkenes using metal complexes (e.g. Mn, Jacobsen, International Patent Application, Publication Number WO/91/14694) is now well established. Recently, non-metallic epoxidates such as peroxides, peracids, dioxiranes and oxaziridines have shown improved enantioselectivity. Notably, the dioxiranes (prepared from the corresponding carbohydrates) of Shi et al (Journal Organic Chemistry, 1997, Vol. 62, no. 8, pp 2328-2329) have shown good enantioselectivity with good product yields.

Asymmetric epoxidations are also known to proceed using complex oxaziridinium salt catalysts which are themselves prepared from complex, fused-ring amines, such as those disclosed by Aggarwal (A) (Chemical Communications, 1996, no. 2, pp 191-192) and Hanquet (B) (Tetrahedron Letters, 1993, Vol. 34, no. 45, pp 7271-7274):

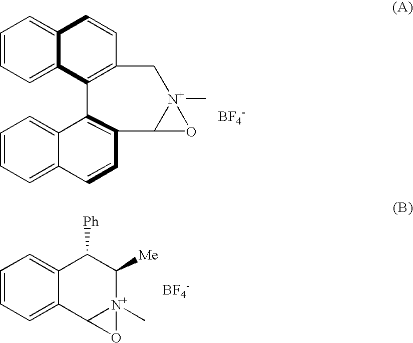

Recently, Armstrong et al (Synlett, 1997, No. 9, pp 1075-1076) has shown that an epoxidation of unfunctionalised alkenes can also be effected in excellent yields using an exocyclic iminium salt (C), derived from pyrrolidine, in the presence of Oxone®.

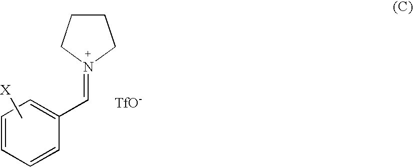

We have now surprisingly discovered that the epoxidation of unfunctionalised alkenes can proceed efficiently and, if required, asymmetrically using a series of simple amines as catalysts and without the need for complex catalysts. Furthermore, the catalytic cycle employed is simple and robust, enabling the use of readily available and cheap reagents as well as environmentally safe solvents.

Accordingly, the invention provides a process for the epoxidation of an alkene, which process comprises reacting an alkene with an oxidising agent in the presence of a catalyst, characterised in that the catalyst is an amine of formula (I):

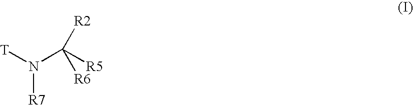

wherein T represents hydrogen or a moiety of formula (a):

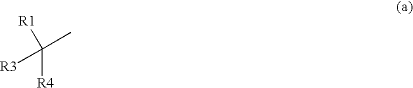

R¹, R², R³, R⁴, R⁵ and R⁶ each independently represents hydrogen, optionally substituted alkyl, an optionally substituted aryl group, heterocyclyl or an optionally substituted aralkyl group wherein substituents for the above mentioned groups are selected from up to three of alkyl, aryl, heterocycyl, hydroxy, alkoxy or a group NR^{s}R^{t} wherein R^{s} and R^{t} each independently represent hydrogen, alkyl or alkylcarbonyl and R⁷ represents hydrogen, alkyl, aryl or aralkyl;

or T represents a moiety (a) wherein R¹ together with R² represents an optionally substituted alkylene chain comprising 2 to 6 carbon atoms the alkylene chain being optionally interrupted with an oxygen atom or a group NR^{p} wherein R^{p} is hydrogen or alkyl, and wherein optional substituents for any carbon atom of the alkylene chain are selected from hydroxy, alkoxy, oxo or a group NR^{s}R^{t} wherein R^{s} and R^{t} each independently represent hydrogen, alkyl or alkylcarbonyl or substituents on any two adjacent carbon atoms of the chain together with the carbon atoms to which they are attached form an alicyclic, aryl or heterocyclic ring; and

R³, R⁴, R⁵, R⁶ and R⁷ are as defined above.

When R¹ together with R² represents an optionally substituted alkylene chain, it is suitably an optionally substituted alkylene chain comprising 2 or 3 carbon atoms.

In one aspect of the compounds of formula (I), T represents a moiety of the above defined formula (a), R¹ together with R² represents an optionally substituted alkylene chain as defined above, R⁵ represents optionally substituted alkyl, an optionally substituted aryl group, heterocyclyl or an optionally substituted aralkyl group wherein substituents for the above mentioned groups are selected from up to three of alkyl, aryl, heterocycyl, hydroxy or alkoxy and R⁷ represents hydrogen, alkyl, aryl or aralkyl and R³, R⁴ and R⁶ each independently represents hydrogen. Suitably, R⁵ represents optionally substituted alkyl, heterocyclyl or an optionally substituted aralkyl group wherein the substituents are selected from an alkyl, aryl, heterocycyl, hydroxy or alkoxy group.

When R⁵ is optionally substituted alkyl, especially optionally substituted C₁₋₆ alkyl, particular substituents include alkoxy, for example methoxy.

When R⁵ is heterocyclyl, particular heterocycyl groups include pyridyl for example a 3-pyridyl group.

Suitably, R⁵ represents an optionally substituted aralkyl group. Preferably, R⁵ represents optionally substituted bisarylalkyl especially bisarylmethyl.

Examples of substituents for R⁵ include hydroxy and alkoxy groups.

Examples of R⁵ include diphenylhydroxymethyl and diphenylmethyl.

A preferred R⁵ group is a diphenylmethyl group.

When R¹ together with R² represents the above defined optionally substituted alkylene chain, R³, R⁴, R⁶ and R⁷ suitably represent hydrogen and R⁵ represents an optionally substituted aralkyl group.

In one particular form of the process, the catalyst is an amine of formula (Ia) or (Ilb):

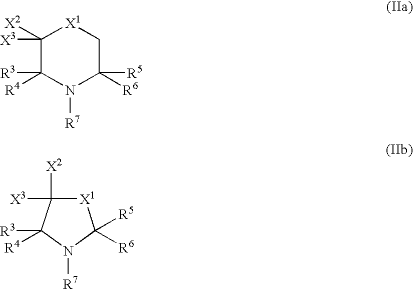

wherein R⁵, R⁶ and R⁷ are as defined above, X¹ is CH₂, O or NX⁴, wherein X⁴ represents hydrogen, alkyl, alkylcarbonyl, alkoxycarbonyl, aryl, aralkyl and either R³ and R⁴ are as defined above, X² independently represents any value of R² and X³ independently represents any value of R³ or X² and R³ each independently represent hydrogen and X³ and R⁴ together with the carbon atoms to which they are attached form an alicyclic or heterocylic ring.

In one preferred form of the process, the catalyst is an amine of formula (IIc):

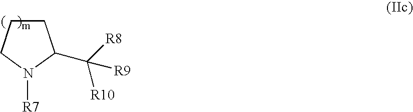

wherein R⁷ is as defined in formula (I) and R⁸ and R⁹ each independently represents an alkyl or, preferably, an aryl group and R¹⁰ represents hydrogen, hydroxy or alkoxy and m is an integer 1 or 2.

Suitably, R⁷ represents hydrogen, C, 6 alkyl or benzyl,

Preferably, R⁷ represents hydrogen.

Suitably, R⁸ represents aryl, especially phenyl.

Suitably, R⁹ represents aryl, especially phenyl.

Suitably, R⁸ and R⁹ each independently represents phenyl.

Suitably, R¹⁰ represents hydrogen.

Suitably, m is an integer 1.

Examples of amines of formula (I) include:

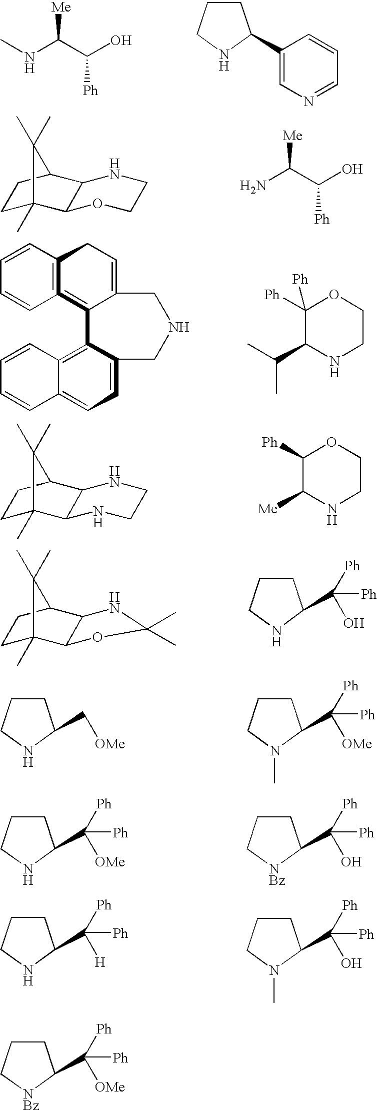

Examples of amines of formulae (IIa) and (IIb) include:

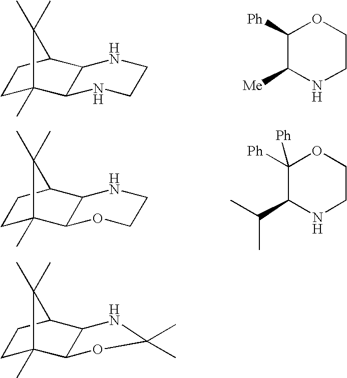

Examples of amines of formula (IIc) include:

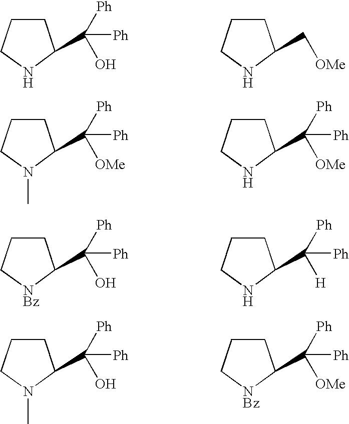

Preferred amines include (S)-(−)-2-(diphenylhydroxymethyl)pyrrolidine and (S)-(−)-2-(diphenylmethyl)pyrrolidine.

The epoxidation reaction may be carried out using any suitable procedure wherein the alkene, preferably a prochiral alkene, the oxidising agent and the compound of formula (I) are allowed to react thereby providing the required epoxide; suitably the reaction is carried out in an organic solvent, such as acetonitrile, or in an organic solvent/water mixture, such as aqueous acetonitrile, at a low to medium-elevated temperature such as a temperature in the range −20° C. to 50° C., preferably at ambient temperature.

As indicated above the epoxidation of the alkene can proceed in an asymmetric manner. This is generally effected by use of an chiral amine catalyst.

Suitable prochiral alkenes include trans-stilbene, 1-methylcyclohex-1-ene, 1-phenylcyclo hex-1-ene, styrene, α-methylstyrene, β-methylstyrene and indene.

Suitable prochiral alkenes also include chromenes.

Additional alkenes include methylenecyclohexene, octahydronapthalene, norbornylene, limonene and carene.

Suitable oxidising agent include conventional oxidising systems, suitably nucleophilic oxidising systems, such as Oxone (KHSO₅), sodium bicarbonate, acetonitrile/water, using methods analogous to those used to prepare known compounds, for example the oxidising agents disclosed by Hanquet et al. (Tetrahedron Letters, 1993, Vol. 34, no. 45, pp 7271-7274).

A suitable oxidising agent is a nucleophilic oxidising agent.

One particularly suitable nucleophilic oxidising agent is provided by a mixture of Oxone® (KHSO₅) and sodium bicarbonate.

Aqueous acetonitrile is an apt reaction solvent when Oxone®/NaHCO₃ is the nucleophilic oxidising agent.

Suitably, the reaction is carried out in the presence of a base such as sodium bicarbonate.

In one preferred form of the process, the oxidation is carried out in the presence of a second base, such as a mild organic base for example pyridine, 2, 6-lutidine and triethylamine.

Suitably the molar ratio of the compound of formula (I) to the prochiral alkene is in the range 100 to 0.01 mol %, preferably from 1 to 10 mol %, for example 5 mol %.

A suitable aryl group is a phenyl group or a naphthyl group.

As used herein, alkyl groups, whether present alone or as part of other groups such as alkoxy or aralkyl groups, are alkyl groups having straight or branched carbon chains, containing 1 to 6 carbon atoms, e.g. methyl, ethyl, n-propyl, iso-propyl, n-butyl, isobutyl or tert-butyl groups.

As used herein, ‘alicyclic group’ includes alicyclic rings of 4 to 12 carbon atoms, especially 4 to 6 carbon atoms.

Suitable heterocyclyl groups include substituted or unsubstituted, single or fused ring aromatic heterocyclyl groups having 5 to 7 ring atoms, suitably 6 ring atoms, said ring atoms comprising up to 4 hetero atoms in each ring, especially 1 or 2, selected from oxygen, sulphur or nitrogen. An example of a heterocyclic ring is a pyridyl ring.

The compounds of formula (I) are generally known, commercially available compounds: For example the compounds of formula (II) wherein the moiety CR⁸R⁹R¹⁰ is diarylmethylhydroxy, may be prepared using methods disclosed by D. J. Mathre (editor I. Shinkai, Organic Synthesis, 1997, Vol. 74, pp 50-71). The compounds of formula (II) wherein the moiety CR⁸R⁹R¹⁰ is diarylmethylmethoxy, are prepared by using methods disclosed by D Enders (Bulletin des Societes Chimiques Belges, 1988, Vol. 97, No. 8-9, pp 691-704). Also, compounds of formula (II) wherein the moiety CR⁸R⁹R¹⁰ is diarylmethyl may be prepared using methods disclosed by D. O'Hagan et al (Tetrahedron: Asymmetry, 1997, Vol. 8, No. 1, pp.149-153).

The following examples illustrate the invention but do not limit it in any way.

### EXAMPLES

### General Epoxidation Procedure Using One Pot Method

To a solution of amine (1) (0.05 mmol) in CH₃CN:H₂O (95:5) (1 ml) were added sequentially alkene 2 (0.5 mmol), NaHCO₃ (5 mmol), Oxone® (0.6 mmol). Within 2 hrs the reaction was diluted with water and extracted with ethyl acetate, dried (MgSO₄), concentrated in vacuo. The crude material was purified (HPLC or flash chromatography) to give the desired epoxide.

### Example 1

### Stoichiometric Epoxidation of 1-Phenylcyclohex-1-ene

To a solution of (S)-(−)-2-(diphenylhydroxymethyl)pyrrolidine 1 (25 mg, 0.1 mmol) in CH₃CN:H₂O (95:5) (1 ml) were added sequentially 1-phenylcyclohex-1-ene 2 (15 □l, 0.11 mmol), NaHCO₃ (90 mg, 1.1 mmol), Oxone® (81 mg, 0.13 mmol). After 1 hr the reaction was diluted with water and extracted with ethyl acetate, (>99% conversion, GC), dried (MgSO₄) and concentrated in vacuo. The crude material was purified via flash chromatography eluting with ethyl acetate:petrol (2;98), to give the desired 1-phenylcyclohex-1-ene oxide 3, 18 mgs, 90%, 72% e.e. (S,S) by GC □-CycloDex 120, 30 m, oven temperature 130° C., injector temperature 200° C., detector temperature 250° C., PSI 20, retention time 12.8 min (S,S) and 13.13 min (R,R).

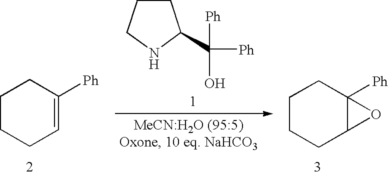

### Example 2

### Catalytic epoxidation of 1-Phenylcyclohex-1-ene

As above except; (S)-(−)-2-(diphenylmethyl)pyrrolidine 4 (1 mg, 0.0042 mmol) in CH₃CN:H₂O (95:5) (0.5 ml), 1-phenylcyclohex-1-ene 2 (67 μl, 0.42 mmol), NaHCO₃ (330 mg, 3.9 mmol), and Oxone® (300 mg, 0.50 mmol). After 40 mins the reaction was diluted and work up as described previously, >99% conversion by GC, 57% e.e.

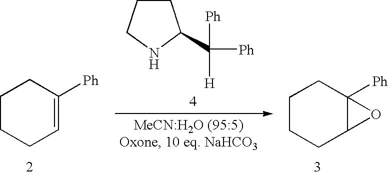

### Example 3

### Catalytic Epoxidation of 1-phenylcyclohex-1-ene.

The procedure of example 1 was repeated except that (S)-(−)-(diphenylmethyl)pyrrolidine 4 (5 mg, 0.021 mmol) in CH₃CN:H₂O (95:5), 1-phenylcyclohex-1-ene 2 (67 W, 0.424 mmol) NaHCO₃ (330 mg, 3.9 mmol), pyridine (17 μl, 0.21 mmol) and Oxone® (500 mg, 0.84 mmol). After 2 h the reaction was diluted with water, extracted with CHCl₃ (>99% conversion by NMR [nitrobenzene as Internal Standard]), dried (NaSO₄) and concentrated in vacuo. The crude material was purified as described previously (example 1) to give 64 mg of 1-phenylcyclohex-l-ene oxide 88%, 57% ee (S,S). The ‘diol’ referenced in Table 1 is a hydrolysis by-product derived from the desired epoxide product.

TABLE 1
Alkene epoxidation using 5 mol % of Amine^{a}

| | | pyrrolidine | | | (S)-2-diphenylmethylpyrrolidine | | |
|---|---|---|---|---|---|---|---|
| | | catalyst | | | catalyst | | |
| | | | epoxide^{d} | | | epoxide^{d} (ee^{e} %) | |
| entry | Alkene | Conv.^{c} % | [isolated] | diol^{d} | Conv.^{c} % | [isolated] | diol^{d} |
| 1 | Methylenecyclohexene | 33 | 29 | 4 | 33 | 29 | 4 |
| 2^{b} | 1-methylcyclohexene | 100^{f} | 90 [81] | 10 | 100^{f} | 90 (15 R,S) [83] | 10 |
| 3 | Octahydronapthalene | 100^{f} | 92 [86] | 8 | 100^{f} | 93 [87] | 7 |
| 4^{b} | Indene | 29 | 19 | 10 | 31 | 20 (25) | 11 |
| 5 | 1-phenylcyclohexene | 59^{f} | 56 [50] | 3 | 100^{f} | 96 (57 S,S) [88] | 4 |
| 6^{b} | α-methylstyrene | 77^{f} | 38 [25] | 38 | 88^{f} | 65 (15 S) [50] | 23 |
| 7 | β-methylstyrene | 15 | 15 | 0 | 21 | 21 (13 S,S) | 0 |
| 8 | Norbornylene | 77 | 74 [71] | 2 | 80 | 74 [69] | 6 |
| 9 | Styrene | 30 | 27 [21] | 2 | 100 | 93 (9 S) [89] | 6 |

^{a}Alkene (0.424 mmol; C_{substrate} = 0.848 mol/L), 5 mol % amine, 2eq Oxone, 10 eq NaHCO₃, 0.5 ml CD₃CN:D₂O (95:5), 0.5 eq. pyridine, 4 h, rt.
^{b}1 eq. pyridine.
^{c}Remainder is alkene.
^{d}Yields determined by ^{f}H-NMR relative to an internal standard (nitrobenzene).
^{e}Enantioselectivities determined by chiral HPLC using an OD chiral column and correlated with literature data.
^{f}Conversion after 2 h.

1. A process for the epoxidation of an alkene, which process comprises reacting an alkene with an oxidising agent in the presence of a catalyst, characterised in that the catalyst is an amine of formula (I): 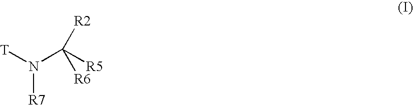
wherein T represents hydrogen or a moiety of formula (a): 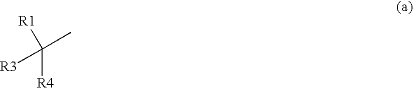
R¹, R², R³, R⁴, R⁵ and R⁶ each independently represents hydrogen, optionally substituted alkyl, an optionally substituted aryl group, heterocyclyl or an optionally substituted aralkyl group wherein substituents for the above mentioned groups are selected from up to three of alkyl, aryl, heterocycyl, hydroxy, alkoxy or a group NR^{s}R^{t} wherein R^{s} and R^{t} each independently represent hydrogen, alkyl or alkylcarbonyl and R⁷ represents hydrogen, alkyl, aryl or aralkyl;
or T represents a moiety (a) wherein R¹ together with R² represents an optionally substituted alkylene chain comprising 2 to 6 carbon atoms the alkylene chain being optionally interrupted with an oxygen atom or a group NR^{p} wherein R^{p} is hydrogen or alkyl, and wherein optional substituents for any carbon atom of the alkylene chain are selected from hydroxy, alkoxy, oxo or a group NR^{s}R^{t} wherein R^{s} and R^{t} each independently represent hydrogen, alkyl or alkylcarbonyl or substituents on any two adjacent carbon atoms of the chain together with the carbon atoms to which they are attached form an alicyclic, aryl or heterocyclic ring; and
R³, R⁴, R⁵, R⁶ and R⁷ are as defined above. A suitable oxidising agent is a nucleophilic oxidising agent.

2. A process according to claim 1, wherein in the compound of formula (I) R¹ together with R² represents an optionally substituted alkylene chain comprising 2 or 3 carbon atoms.

3. A process according to claim 1 or claim 2, wherein in the compound of formula (I) T represents a moiety of the above defined formula (a), R¹ together with R² represents an optionally substituted alkylene chain as defined above, R⁵ represents optionally substituted alkyl, an optionally substituted aryl group, heterocyclyl or an optionally substituted aralkyl group wherein substituents for the above mentioned groups are selected from up to three of alkyl, aryl, heterocycyl, hydroxy or alkoxy and R⁷ represents hydrogen, alkyl, aryl or aralkyl and R³, R⁴ and R⁶ each independently represents hydrogen.

4. A process according to any one of claims 1 to 3, wherein in the compound of formula (I) R⁵ group is a diphenylmethyl group.

5. A process according to claim 1, wherein the compound of formula (I) is an amine of formula (IIa) or (IIb): 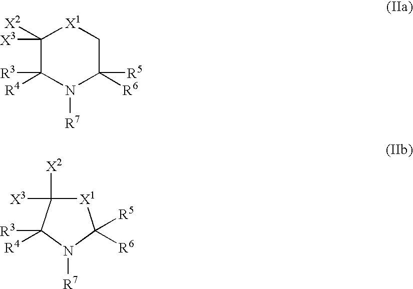
wherein R⁵, R⁶ and R⁷ are as defined in any preceeding claim, X¹ is CH₂, O or NX⁴, wherein X⁴ represents hydrogen, alkyl, alkylcarbonyl, alkoxycarbonyl, aryl, aralkyl and either R³ and R⁴ are as defined in any preceeding claim, X² independently represents any value of R² and X³ independently represents any value of R³ or X² and R³ each independently represent hydrogen and X³ and R⁴ together with the carbon atoms to which they are attached form an alicyclic or heterocylic ring.

6. A process according to claim 1, wherein the compound of formula (I) is an amine of formula (IIc): 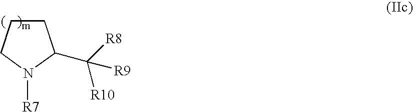
wherein R⁷ is as defined in formula (I) and R⁸ and R⁹ each independently represents an alkyl or, preferably, an aryl group and R¹⁰ represents hydrogen, hydroxy or alkoxy and m is an integer 1 or 2.

7. A process according to any one of claims 1 to 6, wherein in the compound of formula (I) R⁷ represents hydrogen.

8. A process according to any one of claims 1 to 7, wherein in the compound of formula (I) R⁸ and R⁹ each independently represents phenyl.

9. A process according to any one of claims 1 to 8, wherein in the compound of formula (1) R¹⁰ represents hydrogen.

10. A process according to any one of claims 1 to 9, wherein in the compound of formula (I) m is an integer 1.

11. A process according to claim 1, wherein in the compound of formula (I) is selected from the list consisting of:

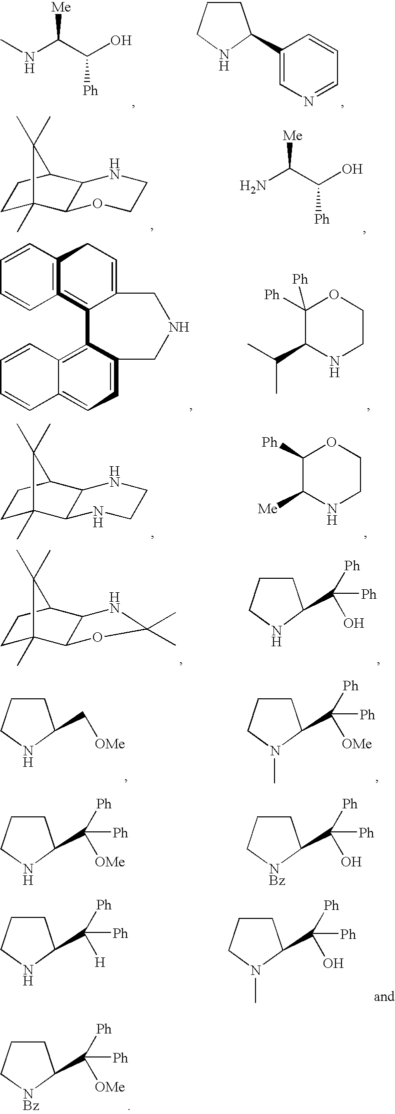

12. A process according to claim 5, wherein in the compound of formulae (IIa) and (IIb)) is selected from the list consisting of:

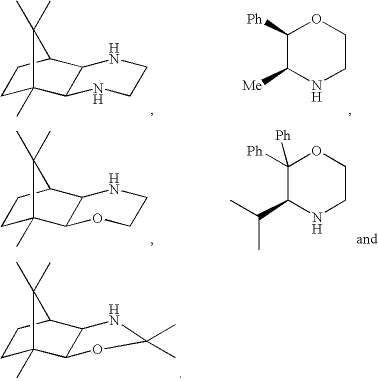

13. A process according to claim 6, wherein in the compound of formulae (IIc) is selected from the list consisting of:

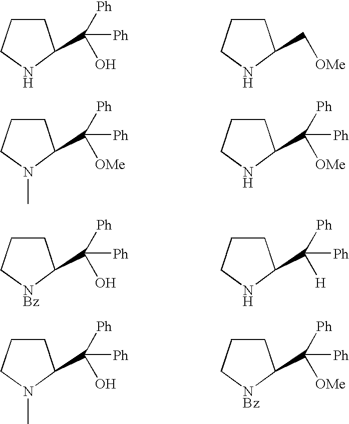

14. A process according to claim 1, wherein in the compound of formula (I) is (S)-(−)-2-(diphenylhydroxymethyl)pyrrolidine or (S)-(−)-2-(diphenylmethyl)pyrrolidine.

15. A process according to claim 1, wherein the oxidising agent is a nucleophilic oxidising agent.

16. A process according to claim 1, wherein the reaction is carried out in the presence of a base.

17. A process according to claim 1, wherein the oxidising agent is a mixture of Oxone® (KHSO₅) and sodium bicarbonate.

18. A process according to claim 1, wherein the oxidation is carried out in the presence of a second base.

19. A process according to claim 18, wherein the second base is pyridine, 2, 6-lutidine or triethylamine.

20. A process according to claim 1, wherein the molar ratio of the compound of formula (I) to the prochiral alkene is in the range of from 1 to 10 mol %.

21. A process according to claim 1, wherein the molar ratio of the compound of formula (I) to the prochiral alkene is 5 mol %.
